(19) United States
(12) Plant Patent Application Publication
(10) Pub. No.: US 2006/0218682 P1
(43) Pub. Date: Sep. 28, 2006

(54) MINIATURE ROSE PLANT 'JENTWELVE'

(50) ROSA HYBRID; MINIATURE

(76) Inventor: Svend Jensen, Christiansfeld (DK)

Correspondence Address: Stefan J. Kirchanski;c/o Liner Yankelevitz Sunshine & Regenstreif LLP, 14th Floor, 1100 Glendon Avenue, Los Angeles, CA, 90024, US

(21) Appl. No.: 11/087,950

(22) Filed: Mar. 23, 2005

(51) Int. Cl. A01H 5/00

(52) U.S. Cl. PLT/122

(57) ABSTRACT

A new miniature rose plant which has abundant, long lasting, red colored flowers and attractive foliage. The variety successfully propagates from softwood cuttings and is suitable for year round production in commercial glass houses as a flowering pot plant. This new and distinct variety has shown to be uniform and stable in the resulting generations from asexual propagation.

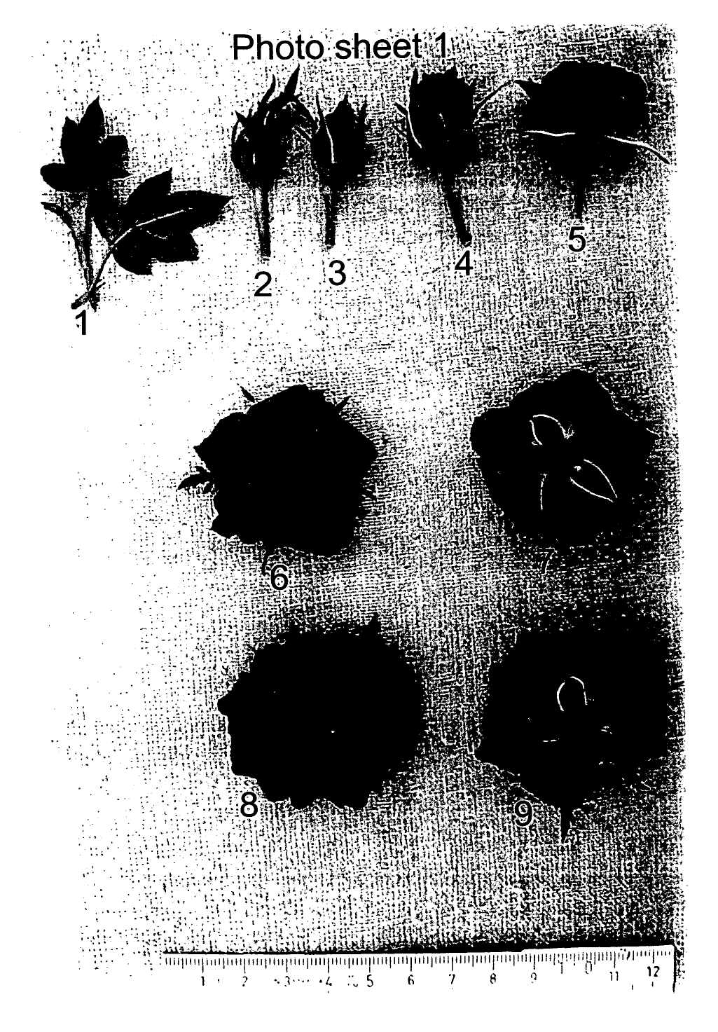

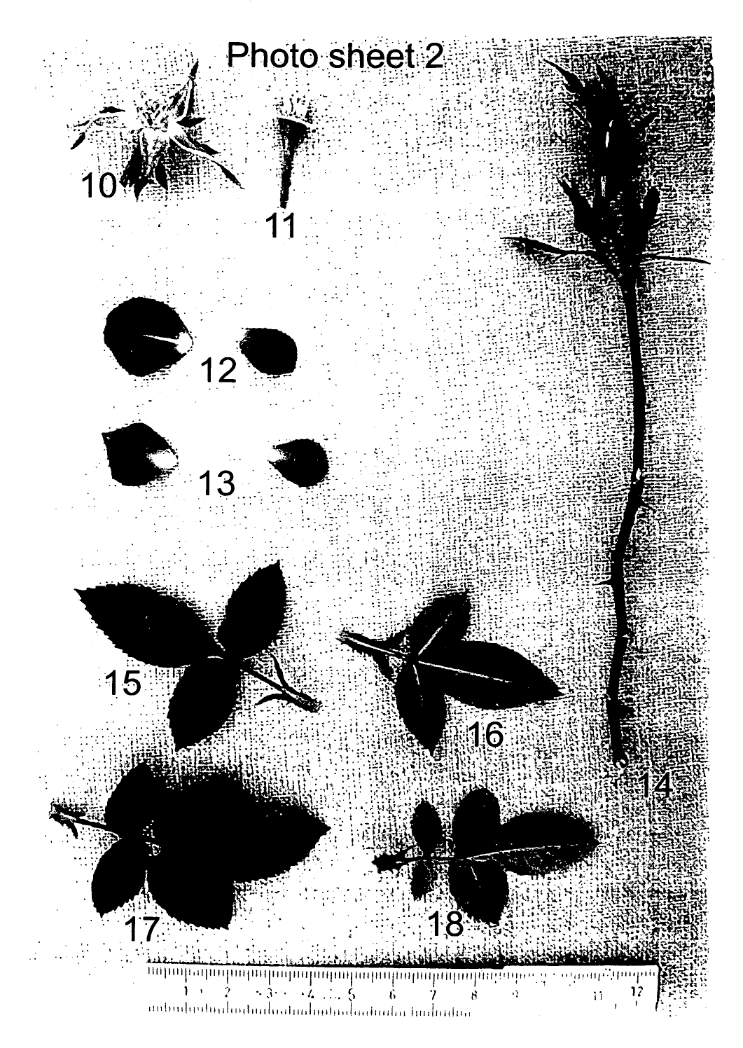

### BACKGROUND OF THE INVENTION

The present invention constitutes a new and distinct variety of miniature rose plant, which was developed by artificially pollinating an unnamed seedling (not patent in the US) with an unnamed seedling (not patent in the US). The two parents were crossed in the summer of 2001 and the resulting seed was sown in December 2001, in a controlled glasshouse environment. Out of these seedlings one seedling was selected, as the new variety and named ‘JENtwelve’. The new rose may be distinguished from its seed parent, an unnamed seedling, by the following combination of characteristics:
1. The unnamed seedling has a breeding background in unnamed seedlings.
2. ‘JENtwelve’ has medium double flowers, while the unnamed seedling has big single flowers.
3. ‘JENtwelve’ has red colored petals, while the unnamed seedling has orange-Red petals.

The new variety may distinguished from its pollen parent, an unnamed seedling created by the same inventor, by the following combination of characteristics:
1. The unnamed seedling has a breeding background in unnamed seedlings.
2. ‘JENtwelve’ has smaller flowers and foliage as compared to the unnamed seedling.
3. ‘JENtwelve’ has red colored petals, while the unnamed seedling has dark pink petals.

### BRIEF SUMMARY OF THE INVENTION

Initial asexual reproduction of ‘JENtwelve’ by cuttings was first done in . The reproduction was conducted in controlled greenhouse environments. Have here proven that the foregoing and all after characteristics and distinctions to come true to form and are established in succeeding propagations. ‘JENtwelve’ is a low and compact miniature rose with medium vigor.

The objective of the hybridization of this rose variety for commercial greenhouse culture was to create a new and distinct variety with:

1. Uniform and abundant flowers with good keepability;

2. Attractive long lasting foliage and compact growth,

3. Year round flowering under glasshouse conditions;

4. Suitability for production from softwood cuttings in pots.;

5. Durable flowers and foliage which make the variety suitable for distribution in the floral industry.

This combination of qualities was not present in previously available commercial cultivars of this type and distinguish ‘JENtwelve’ from all other varieties of which we are aware. The seeds from hybridization were planted in a controlled environment and evaluations were conducted on the resulting plants.

‘JENtwelve’ was selected by Svend Jensen, in his development program in Christiansfeld, Denmark.

### BRIEF DESCRIPTIONS OF THE DRAWINGS

The accompanying color illustrations show as true as is reasonably to obtain in color photographs of this type, the typical characteristics of the buds, flowers, leaves, stems of ‘JENtwelve’. Specifically illustrated in:

### Photo sheet 1

1: Young shoot.

2: Bud before opening the sepals.

3: Bud at the stage of opening the sepals.

4: Bud at the stage of opening the petals.

5: Flower during course of opening.

6: Open flower — plan view — obverse.

7: Open flower — plan view — reverse.

8: Fully open flower — plan view — obverse.

9: Fully open flower — plan view — reverse.

### Photo sheet 2

10: Receptacle showing stamens and pistils.

11: Receptacle showing pistils (stamens and sepals removed).

12: Flower petals, detached — inner surface.

13: Flower petals, detached — outer surface.

14: Bare stem exhibiting thorns and flower attachment.

15: Three leaflets upper side.

16: Three leaflets reverse side.

17: Five leaflets upper side.

18: Five leaflets reverse side.

### DETAILED BOTANICAL DESCRIPTION OF THE VARIETY

The following is a detailed description of the Miniature Rose: Rosa hybrid ‘JENtwelve’.

The following observations, measurements, values and comparisons describe plants grown in glass houses in Christiansfeld, Denmark. The age of the observed plants where 11 to 13 weeks after propagation by cuttings, and produced as a flowering pot plants in container of 10 centimeter in diameter. Environmental conditions in the cultivation area for the observed plants, has been temperatures from 64 to 78 degree Farenheit, and 10 to 20 hours day length with assimilation light. Light levels from 600 foot candle to 6000 foot candle. The relative humidity has been ranging from 50 to 85%. Plants have been regulated with Bonzi to modify the plant shape described

Color references are made using The Royal Horticultural Society (London, England) Colour Chart, 1995, except where common terms of color are used. For a comparison, the nearest existing rose variety is ‘Poulra005’, a rose variety described and illustrated in U.S. Plant Pat. No. 13,108.

CHART 1

| | ‘JENtwelve’ | ‘Poulra005’ |
|---|---|---|
| Petal color, Upper | Red Group | Red Group |
| surface | 43A | 40A |
| Petal color, Reverse | Red Group | Red Group |
| surface | 43A | 47B |
| Petal count | 75-80 | 45-55 |

Parents: Unnamed seedling. Times. Unnamed seedling.
Classification:
   Botanical.—Rosa hybrid.
   Commercial.—Miniature.
Plant:
   Plant growth.—Moderately vigorous. Grows compact upright to bushy. When grown as 10 cm pot plant, the average height of the plant itself is 18 to 20 cm, and average width is 20 cm. When grown as a 15 cm pot plant, the average height of the plant itself is 22 to 27 cm, and average width is 30 cm. Production time is generally 11 to 13 weeks depending on average temperature, light level, and cultural practices.
Stem:
   Color.—Young wood: Yellow-Green Group 146 A-B. Older wood: Yellow-Green Group 147 B.
   Thorns.—Incidence: 1 to 3 thorns per stem. Size: 2-4 mm. Color: Greyed-Red Group 182 B. Shape: Deep concave.
   Surface.—Young wood: Smooth Older wood: Smooth.
   Stem diameter.—2-3 mm.
   Internode length.—20-25 mm.
   Numbers of internodes.—5-6.
Plant foliage: Leaves arranged alternately, compound with 3 to 5 leaflets per leaf, generally symmetrical, abundant, and flat in aspect. Stipules at petiole base.
   Quantity of leaves.—5-6 per lateral branch.
   Leaf size.—Medium 30-60 mm(l). times. 25-40 mm(w), for 5 leaflet.
   Petioles.—Color: Yellow-Green Group 147 A-B, Margins: with prickles Length: 10-12 mm Diameter: about 0.5-1 mm.
   Stipules.—Size: 4-10 mm. Surface: Smooth Color: Yellow-Green Group 147A-B, With intonations of Greyed-Red Group 182B Margins: with prickles.
   Rachis.—Color: Yellow-Green Group 147A, With intonations of Greyed-Red Group 182B Margins: w. prickles. Length: 5 to 15 mm Diameter: 0.5-1 mm.
Leaflets:
   Edge.—Serrated.
   Serration.—Single.
   Shape.—Ovate with acute apex and obtuse base.
   Texture.—Smooth.
   Appearance.—Dull.
   Size.—length: 50 to 70 mm. Width: 40 to 45 mm.
   Color.—Young foliage: Upper surface: Yellow-Green Group 146B With intonations of Greyed-Red Group 179A Lower surface: Greyed-Green Group 191B, With intonations of Greyed-Red Group 179A.
   Color.—Mature foliage: Upper surface: Yellow-Green Group 147A Lower surface: Greyed-Green Group 191A.
Leaf vein color: Upper surface: Yellow-Green Group 147A Reverse surface: Greyed-Green Group 192A.
Inflorescense:
   Blooming habit.—Recurrent.
   Number of flowers.—Generally 3-5 bud per flowering stem.
Peduncle:
   Color.—Yellow-Green Group 146A-B,
   Texture.—Smooth.
   Length.—15-25 mm.
   Strength.—Upright.
Receptacle: Surface: Smooth, glabrous.
   Shape.—Cupped-shaped.
   Size.—h: 5-6 mm w: 5-6 mm.
   Color.—Yellow-Green Group 146a-B.
Sepals:
   Quantity.—5.
   Shape.—Narrowly Ovate w. acute tip.
   Texture.—Leathery.
   Margin.—Foliaceous appendages on 2 of the five sepals.
   Appearance.—Dull.
   Color.—Upper surface: Yellow-Green Group 147A-B, Reverse surface: Greyed-Green Group 191C-D.
   Size.—14 mm(Length), 5 mm(Width).
Buds:
   Size.—10-12 mm (h) 10-12 mm (w)upon opening.
   Shape.—Cupped.
   Color.—at ¼ opening, Red Group 43A.
Flower:
Duration: As a pot plant, flowers last from 15 to 18 days.
   Fragrance.—None.
   Size.—35-45 mm in diameter. Depth: 12-15 mm.
   Form.—Shape of flower when viewed from the side. Up on opening: Cupped Open flower: Flat.
   Color.—
   Petals, upon opening. Upper surface: Red Group 43A, Reverse surface: Red Group 43A.
   Petals after opening.—Upper surface: Red Group 43A, Reverse surface: Red Group 43A. With intonations of White Group 155C.
   Basal Petals spots.—Size: 1-5 mm. Color: White Group 155C.
   General tonality.—On Open flower: Third day: Red Group 43A, Afterwards: Red Group 43A,
Petals:
   Petal reflex.—Outermost petals reflex backwards at opening . Fully open all petals reflex backwards.
   Texture.—Smooth.
   Petal edge.—Entire.
   Petal count.—Approximately 75-80 on the average per flower.
   Petal size.—Length 10-15 mm Width: 10-15 mm.
   Shape.—Outer petals: Round. Inner petals: Ovate Apex: Orbicular, Base: Obtuse to rotundate.
Reproductive organs:
   Stamen number.—Approximately 15-20 on average per flower.
   Stamens length.—2-3 mm.
   Pollen.—Color: Yellow-Orange Group 20D, Abundance: Average.
   Anthers.—Size: 1-1.5 mm Color: Orange-White Group 159D, Shape: Oblong.
   Filaments.—Size: 2-3 mm Color: White Group 155D. With intonations of Greyed-Red Group 182B.
   Pistils number.—Approximately 30 on average per flower.
   Stigmas.—Location: Superior in location to anthers. Color: Green-White Group 157D.
   Styles.—Color: Green-White Group 157D Length: 4 to 5 mm.
Development:
   Vegetation.—Dense.
   Blooming.—Abundant.
   Aptitude to bear fruit.—Poor.
Resistance to diseases: Above average resistance to mildew and Botrytis under normal growing conditions in Christiansfeld, Denmark. Hips/seeds has not been observed due to that the plant has never been grown to the stage of seed development, due to the fact, that the variety is developed for use as a flowering pot plant only.
Winter hardiness & drought/heat tolerance: Due to the fact, that this variety is a potted flowering plant, developed for one time use only(indoor houseplant), the plant is not tested for winter hardiness or drought/heat tolerance

What is claimed is:

1. A new and distinct variety of rose plant of the miniature class, substantially as herein illustrated and described as a distinct and novel rose variety due to its abundant, long lasting, red colored flowers, attractive long lasting foliage, vigorous and compact growth, year round flowering under glasshouse conditions, suitability for production from softwood cuttings in pots, and durable flowers and foliage which make the variety suitable for distribution in the floral industry.
